(19) United States
(12) Patent Application Publication
(10) Pub. No.: US 2009/0240258 A1
(43) Pub. Date: Sep. 24, 2009

(54) DETACHABLE HANDLE FOR IMPLANTABLE ELECTRICAL STIMULATION SYSTEMS AND METHODS OF MAKING AND USING

(75) Inventors: Janusz A. Kuzma, Bayview (AU); Anne Margaret Pianca, Santa Monica, CA (US); Lani A. Smith, Bayview (AU)

Correspondence Address: Boston Scientific Neuromodulation Corp.;c/o DARBY & DARBY P.C., P.O. BOX 770, Church Street Station, NEW YORK, NY, 10008-0770, US

(73) Assignee: Boston Scientific Neuromodulation Corporation, Valencia, CA (US)

(21) Appl. No.: 12/406,885

(22) Filed: Mar. 18, 2009

Related U.S. Application Data
(60) Provisional application No. 61/038,671, filed on Mar. 21, 2008.

(51) Int. Cl. A61B 17/00

(52) U.S. Cl. 606/129

(57) ABSTRACT

A detachable handle includes a handle body that is substantially tubular and defines a hollow center region extending at least a portion of a longitudinal length of the handle body. The handle body is configured and arranged to receive a portion of a stylet handle within the hollow center region and removably attach to the stylet handle. The handle body includes an external gripping surface extending at least a portion of an exterior surface of the handle body.

### CROSS-REFERENCE TO RELATED APPLICATIONS

This Application is a Non-Provisional of Provisional (35 USC 119(e)) application 61/038,671 filed on Mar. 21, 2008, the disclosures of which are herein incorporated by reference in their entirety.

### FIELD

The present invention is directed to the area of implantable electrical stimulation systems and methods of making and using the systems. The present invention is also directed to detachable handles for implantable electrical stimulation systems, as well as methods of making and using the detachable handles.

### BACKGROUND

Implantable electrical stimulation systems have proven therapeutic in a variety of diseases and disorders. For example, spinal cord stimulation systems have been used as a therapeutic modality for the treatment of chronic pain syndromes. Deep brain stimulation has also been useful for treating refractory chronic pain syndromes and has been applied to treat movement disorders and epilepsy. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Moreover, electrical stimulation systems can be implanted subcutaneously to stimulate subcutaneous tissue including subcutaneous nerves such as the occipital nerve.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a detachable handle includes a handle body that is substantially tubular and defines a hollow center region extending at least a portion of a longitudinal length of the handle body. The handle body is configured and arranged to receive a portion of a stylet handle within the hollow center region and removably attach to the stylet handle. The handle body includes an external gripping surface extending at least a portion of an exterior surface of the handle body.

In another embodiment, an electrical-stimulation implantation system includes a lead having a proximal end and a central lumen, a stylet having a stylet handle and an insertion rod, and a detachable handle attachable to a portion of the stylet handle. The insertion rod is insertable into the central lumen of the lead. The detachable handle includes a handle body that is substantially tubular and defines a hollow center region extending at least a portion of a longitudinal length of the handle body. The handle body is configured and arranged to receive a portion of a stylet handle within the hollow center region and removably attach to the stylet handle. The handle body includes an external gripping surface extending at least a portion of an exterior surface of the handle body.

In yet another embodiment, a method for implanting an electrical-stimulation system includes inserting an insertion rod of a stylet with a stylet handle into a central lumen of a lead with a proximal end, attaching a detachable handle to a portion of the stylet handle, and using the detachable handle to guide the lead and coupled stylet into a previously-created surgical incision. The detachable handle includes a handle body that is substantially tubular and defines a hollow center region extending at least a portion of a longitudinal length of the handle body. The handle body is configured and arranged to receive a portion of a stylet handle within the hollow center region and removably attach to the stylet handle. The handle body includes an external gripping surface extending at least a portion of an exterior surface of the handle body.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2 is a schematic view of another embodiment of an electrical stimulation system, according to the invention;

FIG. 3A is a schematic side view of one embodiment of a detachable handle for an electrical-stimulation implantation system, according to the invention;

FIG. 3B is a schematic perspective view of the embodiment of the detachable handle shown in FIG. 3A, according to the invention;

FIG. 3C is a schematic longitudinal cross-sectional view of the embodiment of the detachable handle shown in FIG. 3A, according to the invention;

FIG. 4 is a schematic side view of one embodiment of an electrical-stimulation implantation system, according to the invention;

FIG. 5 is a schematic side view of the embodiment of the electrical-stimulation implantation system shown in FIG. 4 with an insertion rod inserted in a proximal end of a lead, according to the invention;

FIG. 6A is a schematic side view of the embodiment of the electrical-stimulation implantation system shown in FIG. 4 with a stylet handle disposed in a detachable handle, according to the invention;

FIG. 6B is a schematic side view of the embodiment of the electrical-stimulation implantation system shown in FIG. 4 with both a stylet handle and a proximal end of a lead disposed in a detachable handle, according to the invention;

FIG. 7A is a schematic longitudinal cross-sectional view of the embodiment of the detachable handle shown in FIG. 3C with a portion of a stylet handle disposed entirely in a hollow center region of the detachable handle, according to the invention.

FIG. 7B is a schematic longitudinal cross-sectional view of the embodiment of the detachable handle shown in FIG. 3C with a portion of a stylet handle disposed partially in a hollow center region of the detachable handle, according to the invention.

FIG. 7C is a schematic longitudinal cross-sectional view of the embodiment of the detachable handle shown in FIG. 3C with a portion of a stylet handle and a proximal end of a lead each disposed in a hollow center region of the detachable handle, according to the invention.

### DETAILED DESCRIPTION

The present invention is directed to the area of implantable electrical stimulation systems and methods of making and using the systems. The present invention is also directed to detachable handles for implantable electrical stimulation systems, as well as methods of making and using the detachable handles.

Suitable implantable electrical stimulation systems include, but are not limited to, an electrode lead with one or more electrodes disposed on a distal end of the lead and one or more terminals disposed on a proximal end of the lead. Electrode leads include, for example, percutaneous leads and paddle leads. Examples of electrical stimulation systems with electrode leads are found in, for example, U.S. Pat. Nos. 6,181,969; 6,516,227; 6,609,029; 6,609,032; and 6,741,892; and U.S. patent application Ser. Nos. 11/238,240; 11/319,291; 11/327,880; 11/375,638; 11/393,991; and 11/396,309, all of which are incorporated by reference.

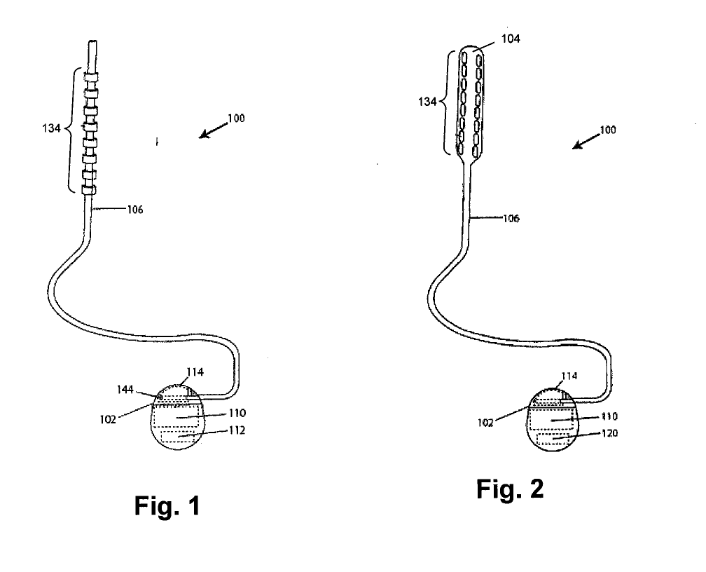

FIG. 1 illustrates schematically one embodiment of a stimulation system 100. The stimulation system includes a control module (e.g., a stimulator or pulse generator) 102 and an array of electrodes 134 disposed at or near the distal end of a lead body 106 forming a percutaneous lead. The percutaneous lead may be isodiametric along the length of the percutaneous lead. The control module 102 typically includes an electronic subassembly 110 and optional power source 120 disposed in a sealed housing 114. The control module 102 typically includes a port 144 into which the proximal end of the lead body 106 can be plugged to make an electrical connection via contacts (not shown in FIG. 1) on the control module 102 and lead body 106. In addition, one or more lead extensions (not shown) can be disposed between the lead and the control module 102 to extend the distance between the control module 102 and the lead body 106. It will be understood that the stimulation system can include more, fewer, or different components and can have a variety of different configurations including those configurations disclosed in the stimulation system references cited herein. For example, as shown in FIG. 2, instead of an array of electrodes disposed at or near the distal end of a lead body 106, a lead may include a paddle body 104, and at least one lead body 106 coupling the control module to the paddle body. Collectively, the paddle body 104 and the lead body 106 form a paddle lead. The paddle body 104 typically includes an array of electrodes 134.

The stimulation system or components of the stimulation system, including one or more of the lead body 106 and the control module 102, are typically implanted into the body. The stimulation system can be used for a variety of applications including, but not limited to, brain stimulation, neural stimulation, spinal cord stimulation, muscle stimulation, and the like.

The electrodes 134 can be made using any conductive material. Examples of suitable materials include metals, alloys, conductive polymers, conductive carbon, and the like, as well as combinations thereof. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of a lead are typically disposed in a non-conductive, biocompatible material including, for example, silicone, polyurethane, polyetheretherketone (PEEK), epoxy, and the like or combinations thereof. A lead may be formed in the desired shape by any process including, for example, molding (including injection molding), casting, and the like. The non-conductive material typically extends from the distal end of the lead to the proximal end. When a paddle body 104 is incorporated into a lead, the non-conductive, biocompatible material of the lead body 106 and paddle body 104 may be the same or different, and the lead body 106 and the paddle body 104 may be a unitary structure or can be formed as two separate structures that are permanently or detachably coupled together.

Conductive contacts are typically disposed at the proximal end of a lead for connection to a corresponding conductive contact in the control module 102 (or to conductive contacts on a lead extension). Conductive wires extend from the conductive contacts to the electrodes 134. Typically, one or more electrodes are electrically connected to a contact. In some embodiments, each contact is only connected to one electrode. The conductive wires may be embedded in the non-conductive material of the lead or can be disposed in one or more lumens extending along the lead. In some embodiments, there is an individual lumen for each conductive wire. In other embodiments, two or more conductive wires may extend through a lumen. There may also be one or more lumens that open at, or near, the distal end of the lead, for example, for infusion of drugs or medication into the site of implantation of the lead.

Leads can be implanted into an implantee using a stylet that includes an insertion rod and a stylet handle. An insertion rod can be slid into the proximal end of a lumen of a lead and used to stiffen the lead in order to facilitate maneuvering of the lead within the implantee. Once an insertion rod is placed within a lead, a health care professional can insert the lead (with the insertion rod) into an incision in the implantee and use the stylet handle to guide the lead to a desired implantation site. Once the insertion rod and the lead are in the desired implantation site, the insertion rod can subsequently be removed from the lead and conductive contacts within the proximal end of the lead can be coupled to corresponding conductive contacts in the control module 102 to establish an electrical connection between the control module 102 and the electrodes 134 at the distal end of the lead.

Implanting an electrical stimulation system can require a high degree of surgical skill. Such skill may be needed to use a stylet handle to safely and precisely maneuver a lead within an implantee to a desired implantation site. Unfortunately, stylet handles are often small and may not provide enough tactile feedback for at least some health care professionals.

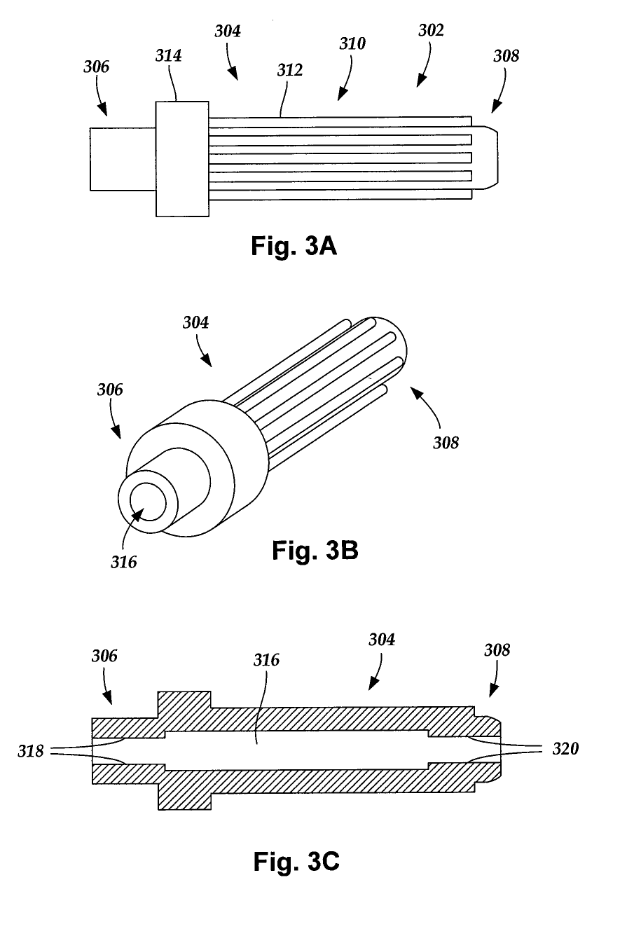

FIG. 3A is a schematic side view of one embodiment of a detachable handle for an electrical-stimulation implantation system, according to the invention. A detachable handle 302 includes a handle body 304 with a first end 306 and a second end 308. The handle body 304 further includes an external gripping surface 310 with a plurality of tactile features, such as tactile feature 312, and an annular feature 314 between the first end 306 and the external gripping surface 310. In the embodiment shown in FIG. 3A, the tactile features are disposed along a longitudinal axis, across a portion of the external gripping surface 310. In other embodiments, the tactile features are disposed along axes other than the longitudinal axis or disposed in multiple axes that either include or do not include the longitudinal axis. In yet other embodiments, the tactile features are of other shapes suitable for providing tactile feedback to a user gripping the external gripping surface 310. In some embodiments, the handle body 304 is sized to accommodate a hand of a user, either customized for individual use or manufactured in one or more sizes designed to fit a wide selection of average-hand-sized users.

FIG. 3B is a schematic perspective view of the embodiment of the detachable handle shown in FIG. 3A, according to the invention. The handle body 304 includes a hollow center region 316 that extends along a longitudinal axis of the handle body 304. In some embodiments, the hollow center region 316 extends the entire longitudinal length of the handle body 304 while, in other embodiments, the hollow center region 316 extends only a portion of the handle body 304. In FIG. 3B, the hollow center region 316 is shown having a cross-sectional shape which is round. In other embodiments, the cross-sectional shape of the hollow center region 316 is a shape other than round. In various embodiments, the cross-sectional shape of the hollow center region 316 may be varied to accommodate the cross-sectional shapes of components received by the hollow center region 316. In some embodiments, various portions of the hollow center region 316 have various different cross-sectional shapes and sizes when, for example, the hollow center region 316 is receiving two or more components with different cross-sectional shapes and/or sizes.

FIG. 3C is a schematic longitudinal cross-sectional view of the embodiment of the detachable handle shown in FIG. 3A, according to the invention. In FIG. 3C, the hollow center region 316 extends the entire longitudinal length of the handle body 304 and includes a first reduced-caliber region 318 at the first end 306 and a second reduced-caliber region 320 at the second end 308. In alternate embodiments, the hollow center region 316 extends through only a portion of the handle body 304. In other alternate embodiments, the first reduced-caliber region 318 is in proximity to the first end 306 and/or the second reduced-caliber region 320 is in proximity to the second end 308. In some embodiments, there are more than two reduced-caliber regions while in other embodiments there is only one reduced caliber region. In various embodiments, the longitudinal length and the caliber of the reduced-caliber regions may vary. For example, in one embodiment the reduced-caliber regions of a handle body collectively comprise approximately half or less of the longitudinal length of the handle body, while in another embodiment the reduced-caliber regions of a handle body collectively comprise approximately one-third or less of the longitudinal length of the handle body. Additionally, in various embodiments, the shape of the reduced-caliber regions may have a cross-sectional shape that is not round. For example, in one embodiment the cross-sectional shape of a reduced-caliber region is oblong. In another embodiment the cross-sectional shape of a reduced-caliber region is rectangular.

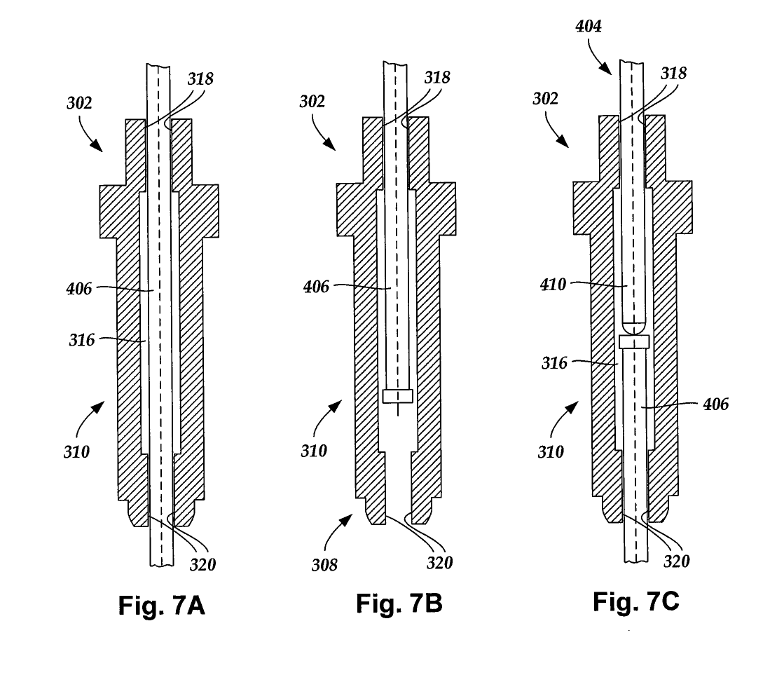

A detachable handle may be formed as a unitary structure or may be formed as two or more separate structures that are permanently or detachably coupled together. A detachable handle can be fabricated in a desired shape by any process, for example, molding, casting, and the like. In some embodiments, a detachable handle is disposed in a compliant material, such as silicone rubber, and the like, which can be used to create an interference fit with components received by a hollow center region, as discussed below, with reference to FIGS. 7A-7C.

A detachable handle can be used to facilitate maneuverability of a lead during implantation of an electrical stimulation system. In one embodiment, a stylet handle is disposed in a detachable handle. In another embodiment, both a stylet handle and a proximal end of a lead are disposed in a detachable handle. Accordingly, the embodiments described above, with relation to FIGS. 3A-3C, may provide an advantage by incorporating a detachable handle, preferably with improved tactile feedback, and also may provide an advantage by making available the option to engage only a stylet handle or to engage both a stylet handle and a lead.

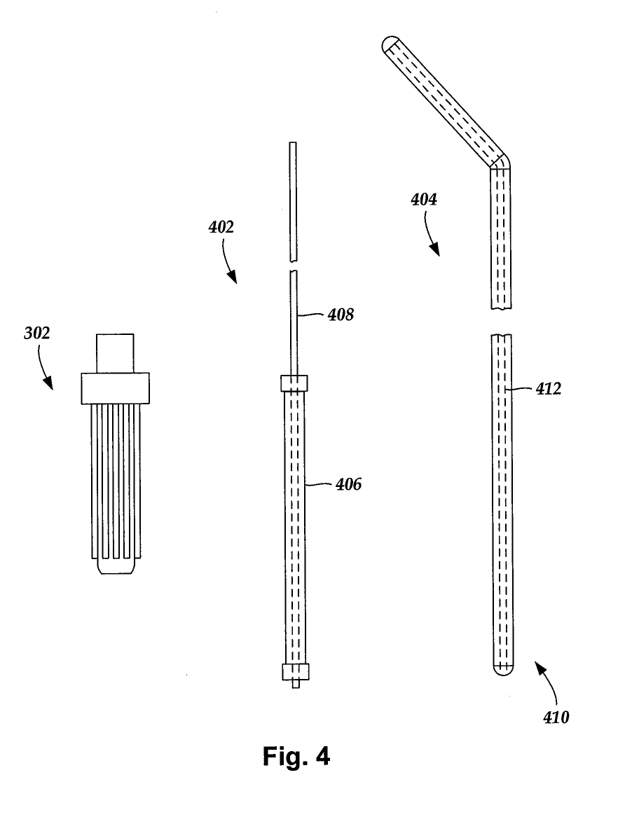

FIG. 4 is a schematic side view of one embodiment of an electrical-stimulation implantation system, according to the invention. An electrical-stimulation implantation system includes a detachable handle 302, a stylet 402, and a lead 404. The stylet 402 includes a stylet handle 406 and an insertion rod 408. The lead 404 includes a proximal end 410 and one or more lumens 412 disposed in at least a portion of a longitudinal length of the lumen 412. In FIG. 4, the insertion rod 408 of the stylet 402 is shown as an elongated rectangle. For clarity of illustration, in several subsequent figures the insertion rod 408 is shown as a line. Also for clarity of illustration, in several subsequent figures the lumen 412 is omitted.

In various embodiments of the present intention, the cross-sectional shape of the stylet 402, the insertion rod 408, the stylet handle 406, the lead 404, and/or the lumen 412 of the lead 404 may be of many different shapes, including, circular, oblong, square, rectangular, triangular, or other suitable shape. In at least some embodiments, the cross-sectional shape of the stylet handle 406 and the proximal end 410 of the lead 404 match the cross-sectional shape of the hollow center region (316 in FIG. 3B) of the detachable handle (302 in FIG. 3A). Additionally, in at least some embodiments, the cross-sectional shape of the lumen 412 matches the cross-sectional shape of the insertion rod 408. In FIG. 4, and in subsequent figures, the lead 404 is shown as a percutaneous lead. In various other embodiments, the lead 404 is a paddle lead (104 in FIG. 2), or some other type of lead.

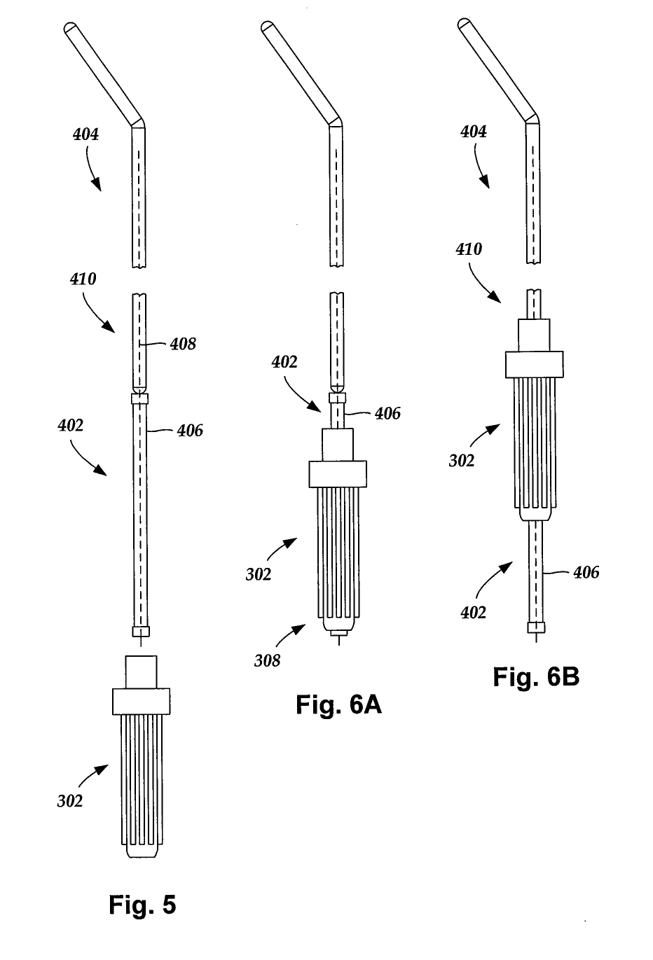

FIG. 5 is a schematic side view of the embodiment of the electrical-stimulation implantation system shown in FIG. 4 with an insertion rod inserted in a proximal end of a lead, according to the invention. The insertion rod 408 of the stylet 402 is partially inserted into the lumen (not shown in FIG. 5) of the lead 404 and the stylet handle 406 is in proximity to the proximal end 410 of the lead 404. The detachable handle 302 is attached to neither the stylet handle 406 nor the proximal end 410 of the lead 404.

In some embodiments, a stylet handle is disposed in a detachable handle while a corresponding lead is not. FIG. 6A is a schematic side view of the embodiment of the electrical-stimulation implantation system shown in FIG. 4 with a stylet handle disposed in a detachable handle, according to the invention. In FIG. 6A, the stylet 402 is disposed in the detachable handle 302. Specifically, the stylet handle 406 is disposed in the hollow center region (316 in FIG. 3B) of the detachable handle 302. In the embodiment shown in FIG. 6A, a portion of the stylet handle 406 extends out of the second end 308 of the detachable handle 302. In other embodiments, the stylet handle 406 does not extend out of the second end 308 of the detachable handle 302.

In other embodiments, both a stylet handle and a proximal end of a lead are disposed in a detachable handle. FIG. 6B is a schematic side view of the embodiment of the electrical-stimulation implantation system shown in FIG. 4 with both a stylet handle and a proximal end of a lead disposed in a detachable handle, according to the invention. In FIG. 6B, the stylet 402 and the lead 404 are disposed in the detachable handle 302. Specifically, the stylet handle 406 and the proximal end 410 of the lead 404 are disposed in the hollow center region (316 in FIG. 3B) of the detachable handle 302. In the embodiment shown in FIG. 6B, a portion of the stylet handle 406 extends out of the second end (308 in FIG. 3A) of the detachable handle 302. In other embodiments, the stylet handle 406 does not extend out of the second end (308 in FIG. 3A) of the detachable handle 302.

Once a desired portion of a stylet handle is disposed in a detachable handle, the detachable handle may be used to create one or more interference fits with the disposed stylet handle. FIG. 7A is a schematic longitudinal cross-sectional view of the embodiment of the detachable handle shown in FIG. 3C with a portion of a stylet handle disposed entirely in a hollow center region of the detachable handle, according to the invention. In FIG. 7A, a portion of the stylet handle 406 is shown disposed entirely in the hollow center region 316 of the detachable handle 302. The two reduced-caliber regions 318 and 320 of the hollow center region 316 contact the stylet handle 406 and provide an interference fit, at least when the detachable handle is squeezed, around each portion of an outer surface of the stylet handle 406 contacting the reduced-caliber regions 318 and 320.

When a user grips the outer gripping surface 310 of the detachable handle 302, the reduced-caliber regions 318 and 320 can be compressed against the portions of the stylet handle 406 contacting the reduced-caliber regions 318 and 320, causing a frictional fastening, or an interference fit, between the detachable handle 302 and the stylet handle 406. In alternate embodiments, the stylet handle 406 is disposed in the hollow center region 316 so as to only contact one reduced-caliber region or to contact more than two reduced-caliber regions.

In one embodiment, a detachable handle is fabricated from a material that is compliant enough, such as silicone rubber, to form an interference fit by the compression created by application of a grip, or at least a squeeze, by a user to the outer gripping surface 310 of the detachable handle 302. Typically, the tighter a user's grip around the outer gripping surface 310, the greater the compression of the detachable handle 302 against the stylet handle 406 and, consequently, the stronger the interference fit. The strength of an interference fit can also be affected by the types of materials used to fabricate the outer surface of the stylet handle, as well as the types of materials used to fabricate a detachable handle. Thus, variable-strength interference fits can be created by fabricating detachable handles with materials of variable compliances.

FIG. 7B is a schematic longitudinal cross-sectional view of the embodiment of the detachable handle shown in FIG. 3C with a portion of a stylet handle disposed partially in a hollow center region of the detachable handle, according to the invention. In FIG. 7B, the stylet handle 406 is shown contacting the reduced-caliber region 318 of the detachable handle 302 without contacting the reduced-caliber region 320 or extending out of the second end 308 of the detachable handle 302. An interference fit may be created between the detachable handle 302 and the stylet handle 406 by a user gripping the outer gripping surface 310, as described above, with reference to FIG. 7A. In alternate embodiments, the stylet handle 406 is placed in contact with more than one reduced-caliber region without extending out of the second end 308 of the detachable handle 302.

In other embodiments, once a desired portion of a stylet handle and a proximal end of a lead each are disposed in a detachable handle, the detachable handle may be used to create one or more interference fits with both the engaged stylet handle and the proximal end of the lead. FIG. 7C is a schematic longitudinal cross-sectional view of the embodiment of the detachable handle shown in FIG. 3C with a portion of a stylet handle and a proximal end of a lead each disposed in a hollow center region of the detachable handle, according to the invention. In FIG. 7C, the stylet handle 406 and the proximal end 410 of the lead 404 both are disposed in the hollow center region 316 of the detachable handle 302. The stylet handle 406 is contacting the reduced-caliber region 320, while the proximal end 410 of the lead 404 is contacting the reduced-caliber region 318. Consequently, when the outer gripping surface 310 is gripped, or at least squeezed, by a user, an interference fit may be created with both the stylet handle 406 and the proximal end 410 of the lead 404, as discussed above, with reference to FIG. 7A. In some embodiments, the stylet handle 406 and/or the proximal end 410 of the lead 404 are disposed so as to be in contact with more than one reduced-caliber region. In some other embodiments, the proximal end 410 of the lead 404 contacts one or more reduced-caliber regions, while the stylet handle 406 is disposed so as to not contact a reduced-caliber region.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A detachable handle comprising:
a handle body that is substantially tubular and defines a hollow center region extending at least a portion of a longitudinal length of the handle body, the handle body configured and arranged to receive a portion of a stylet handle within the hollow center region and removably attach to the stylet handle, the handle body comprising an external gripping surface extending at least a portion of an exterior surface of the handle body.

2. The detachable handle of claim 1, wherein the hollow center region has a cross-sectional shape that is round.

3. The detachable handle of claim 2, wherein the hollow center region includes at least one reduced-caliber region.

4. The detachable handle of claim 3, wherein the hollow center region is configured and arranged to promote an interference fit with the stylet handle when a portion of the stylet handle is placed in contact with at least one reduced-caliber region and the external gripping surface is grasped.

5. The detachable handle of claim 3, wherein the hollow center region is also configured and arranged to receive a proximal end of a lead.

6. The detachable handle of claim 5, wherein the hollow center region is configured and arranged to promote an interference fit with the proximal end of the lead when the proximal end of the lead is placed in contact with at least one reduced-caliber region and the external gripping surface is grasped.

7. The detachable handle of claim 3, wherein the hollow center region is configured and arranged to promote an interference fit with a portion of the stylet handle when a user squeezes the external gripping surface.

8. The detachable handle of claim 1, wherein the handle body is a unitary structure.

9. The detachable handle of claim 1 wherein the handle body is formed by molding.

10. The detachable handle of claim 1, wherein the handle body is fabricated from a compliant material.

11. The detachable handle of claim 1, wherein the handle body is fabricated from silicone rubber.

12. The detachable handle of claim 1, wherein the external gripping surface includes a plurality of tactile features.

13. An electrical-stimulation implantation system comprising:
a lead having a proximal end and a central lumen;
a stylet having a stylet handle and an insertion rod, the insertion rod insertable into the central lumen of the lead; and
a detachable handle attachable to a portion of the stylet handle, the detachable handle comprising a handle body that is substantially tubular and defines a hollow center region extending at least a portion of a longitudinal length of the handle body, the handle body configured and arranged to receive a portion of a stylet handle within the hollow center region and removably attach to the stylet handle, the handle body comprising an external gripping surface extending at least a portion of an exterior surface of the handle body.

14. The electrical-stimulation implantation system of claim 13, wherein the detachable handle is configured and arranged for receiving a portion of the stylet handle by sliding the detachable handle over a portion of the stylet handle.

15. The electrical-stimulation implantation system of claim 13, wherein the hollow center region is also configured and arranged to receive the proximal end of the lead.

16. The electrical-stimulation implantation system of claim 15, wherein the detachable handle is also attachable to the proximal end of the lead.

17. The electrical-stimulation implantation system of claim 16, wherein the detachable handle is attachable to the proximal end of the lead by sliding the detachable handle over the proximal end of the lead.

18. A method for implanting an electrical-stimulation system, the method comprising:
inserting an insertion rod of a stylet with a stylet handle into a central lumen of a lead with a proximal end;
attaching a detachable handle to a portion of the stylet handle, the detachable handle comprising a handle body that is substantially tubular and defines a hollow center region extending at least a portion of a longitudinal length of the handle body, the handle body configured and arranged to receive a portion of a stylet handle within the hollow center region and removably attach to the stylet handle, the handle body comprising an external gripping surface extending at least a portion of an exterior surface of the handle body; and
using the detachable handle to guide the lead and coupled stylet into a previously-created surgical incision.

19. The method of claim 18, wherein attaching the detachable handle to a portion of the stylet handle comprises sliding the stylet handle into the hollow center region of the handle body.

20. The method of claim 19, wherein attaching the detachable handle to a portion of the stylet handle further comprises a firm grip of the external gripping surface by a user.
